(19) United States
(12) Patent Application Publication
(10) Pub. No.: US 2021/0221000 A1
(43) Pub. Date: Jul. 22, 2021

(54) SYSTEMS AND METHODS FOR LIFESAVING TRAUMA STABILIZATION MEDICAL TELEPRESENCE OF A REMOTE USER

(71) Applicant: MONROE SOLUTIONS GROUP INC., Stoney Creek (CA)

(72) Inventors: Imants Dan JUMIS, Mississauga (CA); Abdulmotaleb EL SADDIK, Ottawa (CA); Haiwei DONG, Ottawa (CA); Yang LIU, Ottawa (CA)

(21) Appl. No.: 17/226,394

(22) Filed: Apr. 9, 2021

Related U.S. Application Data
(63) Continuation of application No. 15/995,483, filed on Jun. 1, 2018, now abandoned.
(60) Provisional application No. 62/513,664, filed on Jun. 1, 2017.

(51) Int. Cl. B25J 9/16; G06T 19/00; A61B 34/35; A61B 5/00; G16H 40/67; G16H 80/00; G16H 20/40

(52) U.S. Cl. CPC B25J 9/1689; G06T 19/006; A61B 34/35; A61B 90/37; G16H 40/67; G16H 80/00; G16H 20/40; A61B 5/0022

(57) ABSTRACT

Methods and systems for providing a lifesaving trauma stabilization medical telepresence to a remote user are presented. A data connection between a lifesaving trauma stabilization helmet associated with a first user in a first location and a display device in a second location is established. First video data and first audio data are collected and transmitted to the display device. The first video data and the first audio data are output on the display device to the remote user. Contextual information for the first user is collected from the remote user. The contextual information collected from the remote user is transmitted to the lifesaving trauma stabilization helmet. The contextual information is presented to the first user as haptic feedback using a haptic output device comprising two groups of three vibrating elements having unique tones integrated to opposing lateral sides of the lifesaving trauma stabilization helmet.

### TECHNICAL FIELD

The present disclosure relates generally to medical emergency management, and more particularly to systems and methods for lifesaving trauma stabilization.

### BACKGROUND

First responders, such as paramedics and military medics, are often the first persons to arrive on the scene of catastrophic events and must act quickly and decisively to save lives and minimize injury with lifesaving trauma stabilization. However, the medical training provided to first responders is often limited: many first responders are provided with general training, but lack the advanced doctor-level medical expertise to deal with specific occurrences and/or life threatening types of critical trauma cases. In addition, in most jurisdictions, regulations are placed on the types of procedures which can be performed by first responders on their own in the field, instead requiring that a doctor be present or provide medical oversight guidance to first responders, for instance in life threatening trauma-related situations. Trauma victims need to receive definitive medical care within the “golden hour” after injury, or else they may die unnecessarily.

Although it is possible to connect first responders with doctors via traditional communication platforms, the currently available methods suffer from various disadvantages. For instance, currently available methods typically require a user to hold a device in one or both hands, which may hamper the manual dexterity of the user. Additionally, some communication platforms offer only voice-based communication, which limits the information which can be provided to the doctor. Also current methods do not provide the visual detail resolution and depth perception required for medical oversight of lifesaving procedures which can be oversight guided by remote trauma surgeons in critical cases such as broken ribs in flail chest and punctures close to carotid or femoral arteries.

It would be beneficial to provide a system for connecting first responders with doctors which ameliorates or eliminates some or all of the above-noted shortcomings.

### SUMMARY

In accordance with a broad aspect, there is provided a method for providing a lifesaving trauma stabilization medical telepresence to a remote user. The method comprises: establishing a data connection between a lifesaving trauma stabilization helmet associated with a first user in a first location and a display device in a second location, the first location being remote from the second location; collecting, by a first input device communicatively integrated to the lifesaving trauma stabilization helmet, first video data and first audio data; transmitting, to the display device by the data connection, the first video data and the first audio data acquired by the input device; outputting the first video data and the first audio data on the display device to the remote user; in response to outputting the first video data and the first audio data, collecting contextual information for the first user from the remote user using a second input device located in the second location; transmitting, to the lifesaving trauma stabilization helmet by the data connection, the contextual information collected from the remote user; and presenting the contextual information to the first user as haptic feedback using a haptic output device comprising two groups of three vibrating elements having unique tones integrated to opposing lateral sides of the lifesaving trauma stabilization helmet, wherein presenting the contextual information to the first user as haptic feedback comprises producing a vibration pattern by causing at least some vibrating elements of the two groups of three vibrating elements to produce vibrations.

In accordance with another broad aspect, there is provided a system for providing lifesaving trauma stabilization. The system comprises: a processor; a memory storing computer-readable instructions; a network interface; and a lifesaving trauma stabilization helmet configured for mounting to a head of a first user. The lifesaving trauma stabilization helmet comprises at least one camera configured to capture first video data; at least one microphone configured to capture first audio data; at least one speaker; and a haptic output device comprising two groups of three vibrating elements having unique tones integrated to opposing lateral sides of the helmet. The computer-readable instructions are executable by the processor for: transmitting, by the network interface, the first video data and the first audio data to a remote display device configured to output the first video data and the first audio data to the remote user in a second location, the first location being remote from the second location; obtaining, by the network interface, contextual information for the first user, the contextual information collected from the remote user using a remote input device located in the second location; and in response to obtaining the contextual information for the first user, presenting the contextual information to the first user as haptic feedback by causing at least some vibrating elements of the two groups of three vibrating elements to produce vibrations.

### BRIEF DESCRIPTION OF THE DRAWINGS

The invention will be described in greater detail with reference to the accompanying drawings, in which:

FIG. 1 is an illustration of an example lifesaving trauma stabilization helmet for providing lifesaving trauma stabilization medical telepresence;

FIG. 2 is a block diagram of an example lifesaving trauma stabilization medical telepresence system;

FIG. 3 is a flowchart illustrating an example embodiment of a process for providing lifesaving trauma stabilization via a remote user;

FIG. 4 is a communication diagram for the lifesaving trauma stabilization system of FIG. 2;

FIG. 5 is a schematic diagram of an example embodiment of a computing system for implementing the processes of FIG. 3;

FIG. 6 is a schematic diagram of an example implementation of the lifesaving trauma stabilization system of FIG. 2.

### DETAILED DESCRIPTION

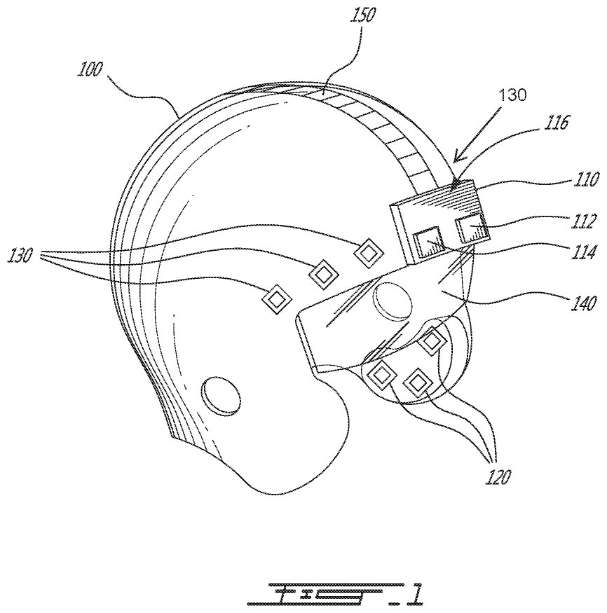

With reference to FIG. 1, there is shown an embodiment of a headgear device 100 configured to provide lifesaving trauma stabilization medical telepresence. The headgear device 100 may be worn on or around a head of a user, for instance a first responder, or is otherwise retained on a portion of the head of the user. Other embodiments are contemplated in which some of all of the device 100 are found in locations other than the user's head. The headgear device 100 may include one or more of a helmet, a headband, a hat, a cap, a pair of glasses, one or more contact lenses, one or more earphones, headphones, earbuds, and the like, or any suitable combination thereof. In some embodiments, the headgear device 100 is a lifesaving trauma stabilization helmet. The headgear device 100 is configured for capturing various data from the environment in which the user of the headgear device 100 is located, and for replaying communications received from a remote user in a remote location, as described in greater detail hereinbelow. In some embodiments, the headgear device 100 includes an audio/video (AV) capture device 110, one or more speakers 120, a haptic system 130, a head-up display (HUD) 140, and a communications interface 150. In certain example implementations, the AV capture device 110, speakers 120, haptic system 130, the HUD 140, and communications interface 150 are integrated within the structure of the headgear device 100. For instance, the headgear device 100 may be embodied as a helmet, and one or more of the AV capture device 110, speakers 120, haptic system 130, the HUD 140, and communications interface 150 may be embedded within the structure of the helmet, or otherwise integrated therein.

The AV capture device 110 may include one or more cameras 112, 114, and a microphone 116. The cameras 112, 114, are configured for capturing video data, and the microphone 116 is configured for capturing audio data in the vicinity of headgear device 100. In some embodiments, the cameras 112, 114 are configured for cooperating to capture stereoscopic video data, which is also known as three-dimensional video data. In some embodiments, the video data may be captured in a medical-grade high-definition format, including stereoscopic or three-dimensional video data, using any suitable video encoding technique. In some embodiments, the AV capture device 100 employs video and/or audio compression techniques to reduce the amount of bandwidth required to transmit the video data and audio data in real time, as will be described in greater detail hereinbelow. For instance, the video data and audio data may be transmitted over a bandwidth of less than 2 Mbps (megabytes per second). In addition, the video data and the audio data captured by the AV capture device 100 may be synchronized and packaged into a common data stream for transmission.

The cameras 112, 114 may be any suitable type of camera, and in some embodiments are digital cameras substantially similar to those used, for example, in smartphones. In some embodiments, the cameras 112, 114 are binocular cameras, and may be provided with any suitable zoom functionality. In some embodiments, the cameras 112, 114 are equipped with motors or other driving mechanisms which can be controlled to adjust a position of one or more of cameras 112, 114 on the headgear device 100, a direction of the cameras 112, 114, a zoom level of the cameras 112, 114, and/or a focal point of the cameras 112, 114. In some embodiments, the headgear device 100 is configured to receive camera control data from the remote user for moving the cameras 112, 114.

In some embodiments, the AV capture device 110 has a single camera, for example camera 112. In embodiments with one camera, the camera 112 may be placed in a substantially central location on the headgear device 100, for example aligned with a longitudinal axis of the headgear device 100, or may be offset from the longitudinal axis. For example, the camera 112 may be placed on a side of the headgear device 100, thereby aligning the camera with an eye of the user when the user wears the headgear device 100. In embodiments where the AV capture device 100 has two cameras 112, 114, the cameras 112, 114 may be placed equidistant from the longitudinal axis of the headgear device 100. The cameras 112, 114 may be located close to a central location on the headgear device 100, or may be spaced apart. In some embodiments, the headgear device 100 includes additional cameras beyond the cameras 112, 114, which can be distributed over the headgear device 100 in any suitable configuration.

The microphone 116 can be any suitable analog or digital microphone. In some embodiments, the microphone 116 is an array of microphones, which are distributed over the headgear device 100 in any suitable arrangement. For example, the array of microphones 116 may be used to collect audio data that can be processed to provide surround-sound. In some embodiments, the AV capture device 110 is a single device which combines or integrates the cameras 112, 114 and the microphone 116, for example as part of a single circuit board.

The speakers 120 are configured for providing playback of audio data received from a remote user at a remote location. The speakers 120 may be a single speaker or a plurality of speakers, and may be arranged at suitable locations about the headgear device 100. In some embodiments, the speakers 120 may be located proximal to one or more of the user's ears. In some embodiments, one or more first speakers are located on an inside wall of a first side of the headgear device 100, and one or more second speakers are located on an inside wall of a second side of the headgear device 100. In another embodiment, the speakers 120 are provided by way of one or more devices for inserting in ear canals of the user of the headgear device 100, for example earbuds. In a further embodiment, the speakers 120 include a plurality of speakers 120 which are arranged within the headgear device 100 to provide a surround-sound like experience for the user.

Additionally, the headgear device 100 may include haptic system 130. The haptic system 130 is configured to provide various contextual information to the user of the headgear device 100 using haptic feedback, including vibrations, nudges, and other touch-based sensory input, which may be based on data received from the remote user. Put differently, the haptic system 130 may be used to simulate tactile stimuli for presentation to the user of the headgear device 100. The haptic feedback can be provided by one or more vibrating elements. As depicted, haptic system 130 includes three vibrating elements on the side of the headgear device 100 visible in FIG. 1—three other vibrating elements may be located on the opposite side of the headgear device 100. It should be noted that the haptic system 130 can include more than three or less than three vibrating elements which can be distributed as appropriate over the headgear device 100. In some embodiments, the headgear device 100 includes at least four vibrating elements which are positioned at front, rear, and side locations of the headgear device 100. In some embodiments, the vibrating elements can be caused to vibrate to indicate to the user of the headgear device 100 that the user should move in a certain direction which corresponds to the vibration of the vibrating elements. For example, causing the front vibrating element to vibrate may indicate to the user that they should move their head back. Alternatively, causing the front vibrating element to vibrate may indicate to the user that they should move their head forward. In another example, causing all the vibrating elements to vibrate may indicate to the user that there is an emergency or dangerous situation. Other information may be conveyed through haptic system 130.

In one example implementation, the haptic system 130 includes a total of six vibrating elements, with three vibrating elements located on the left side of the headgear device 100, and three vibrating elements located on the right side of the headgear. The haptic system 130 is controllable to present contextual information to the user of the headgear device 100 using the aforementioned haptic feedback, which in this example implementation includes controlling each of the vibrating elements independently of one another. For instance, the haptic system 130 can be controlled to present patterns of haptic feedback via the six vibrating elements. Haptic feedback patterns are formed by one or more of the six vibrating elements producing haptic feedback (i.e., vibrating). In some instances, each of the six vibrating elements may be configured for producing haptic feedback in a unique tone: a first one of the vibrating elements may produce vibrations at a first frequency different from the frequency of vibration of the other vibrating elements. In this example implementation, six individually-controllable vibrating elements can be used to produce up to 6 factorial (6!), or 720, unique patterns. It should be understood that other example implementations, in which the haptic system 130 includes a different count of vibrating elements, are also considered. For instance, an implementation in which the haptic system 130 includes eight individually-controllable vibrating elements (e.g., 4 on each side) could be used to produce up to 8 factorial (8!), or 40,320, unique patterns. The use of the vibrating elements of the haptic system 130 to produce haptic feedback patterns can provide a full and rich signaling capability even in harsh environments, which may assist in providing lifesaving trauma stabilization medical telepresence using limited bandwidth and high frequency.

The headgear device 100 further includes interface 150. The interface 150 is configured for establishing a data connection between the headgear device 100 and various other electronic components, as is discussed hereinbelow. The interface 150 may be communicatively coupled to the various components of the headgear device 100, including the AV capture device 110 for providing recorded video data and local audio data from the AV capture device 110 to other components. In addition, the interface 150 may be communicatively coupled to the speakers 120 and the haptic system 130 for providing received remote audio data and haptic data to the speakers 120 and the haptic system 130, respectively. In some embodiments, the interface 150 is a wired interface which includes wired connections to one or more of the AV capture device 110, the speakers 120, and the haptic system 130. In other embodiments, the interface 150 is a wireless interface which includes wireless connections to one or more of the AV capture device 110, the speakers 120, and the haptic system 130. For example, the interface 150 uses one or more of Bluetooth™, Zigbee™, and the like to connect with the AV capture device 110, the speakers 120, and the haptic system 130. In some embodiments, the interface 150 includes both wireless and wired connections.

In some embodiments, the headgear device 100 may include the HUD 140 which can include one or more screens and/or one or more visors. The HUD 140 is configured for displaying additional information to the user of the headgear device 100, for example a time of day, a location, a temperature, or the like. In some embodiments, the HUD 140 is configured to display information received from the remote user.

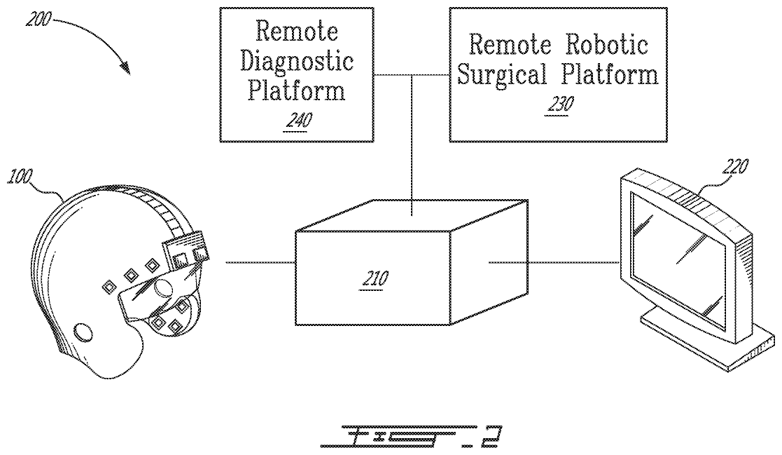

With reference to FIG. 2, the headgear device 100 is part of a lifesaving trauma stabilization medical telepresence system 200 which includes the headgear device 100, a server box 210, and a display device 220. The server box 210 is configured for establishing a data connection between the headgear device 100, for example via the interface 150, and the display device 220. In some embodiments, the telepresence system 200 further includes a remote robotic surgical platform 230 and/or a remote diagnostic platform 240, which may be connected to the server box 210 via any suitable wired or wireless means. In some other embodiments, the remote robotic surgical platform 230 and/or the remote diagnostic platform 240 are connected to one or more of the headgear device 100, the server box 210, and the display device 220 substantially directly or indirectly, as appropriate, using any suitable wired or wireless means, including cellular connections, Wi-Fi connections, and the like.

The display device 220 is configured for displaying the video data and the local audio data collected by the AV capture device 110, and for collecting the remote audio data and the haptic data from the remote user, as discussed in greater detail herein below. In some embodiments, the remote user is a doctor, physician, or trauma surgeon. In some embodiments, at least part of the data connection is established over the Internet.

The data connection between the headgear device 100 and the display device 220 may be a wired connection, a wireless connection, or a combination thereof. For example, some or all of the data connection between the headgear device 100 and the server box 210 may be established over a wired connection, and the data connection between the server box 210 and the display device 220 may be established over a wireless connection. In another example, the data collected by the AV capture device 110 is provided to the server box 210 over a wired connection, and the data sent to the speakers 120 and the haptic system 130 is received over a wireless connection. Wired connections may use any suitable communication protocols, including but not limited to RS-232, Serial ATA, USB™, Ethernet, and the like. Wireless connections may use any suitable protocols, such as WiFi™ (e.g. 802.11a/b/g/n/ac), Bluetooth™, Zigbee™, various cellular protocols (e.g. EDGE, HSPA, HSPA+, LTE, 5G standards, etc.) and the like. It should be noted that the different types of data provided to the display device from the headgear device 100 may require different bandwidths for transmission. For instance, the transmission of data to the haptic system 130 may require a smaller amount of bandwidth than is required for transmission of data obtained by the AV capture device 110.

The server box 210 can be any suitable computing device or computer configured for interfacing with the headgear device 100 and the display device 220 and for facilitating the transfer of audio, video, and haptic data between the headgear device 100 and the display device 220, as well as any other data, including data for the HUD 140, control data for moving the cameras 112, 114, and the like. In some embodiments, the server box 220 can be implemented as a mobile application on a smartphone or other portable electronic device. In other embodiments, the server box 210 is a portable computer, for instance a laptop computer, which may be located in a backpack of the user. In further embodiments, the server box 210 is a dedicated computing device with application-specific hardware and software, which is attached to a belt or other garment of the user. In still further embodiments, some or all of the server box is integrated in the headgear device 100.

In some embodiments, the server box 210 is provided with controls which allow the user to control the operation of the server box 210. For example, the server box 210 may include a transmission switch which determines whether or not the server box performs transmission of the video data and local audio data collected by the headgear device 100. In some embodiments, the server box 210 includes a battery or other power source which is used to provide power to the headgear device 100, and the transmission switch also controls whether the battery provides power to the headgear device 100. In another example, the server box 210 includes a variable quality control which allows the user to adjust the quality of the video data and local audio data transmitted to the display device 220. Still other types of controls for the server box 210 are contemplated.

The display device 220 is configured for receiving the video data and the local audio data from the headgear device 100 (via server box 210) and for performing playback of the video data and the local audio data. This includes displaying the video data, for example on a screen or other display, and outputting the local audio data via one or more speakers or other sound-producing devices. In some embodiments, the display device performs playback of only the video data. The display device 220 also includes one or more input devices via which the remote user (e.g. a doctor, trauma surgeon, etc.) can use to provide remote audio data and/or the haptic data for transmission to the headgear device 100, as well as any additional data, for example the data for the HUD 140 and/or control data for moving the cameras 112, 114. The display device 220 may further include a processing device for establishing the data connection with the headgear device 100, including for receiving the video data and the local audio data, and for transmitting the remote audio data and the haptic data.

The remote robotic surgical platform 230 provides various robotic equipment for performing surgery, including robotic arms with various attachments (scalpels, pincers, and the like), robotic cameras, and any other suitable surgery-related equipment. The remote robotic surgical platform 230 can be controlled remotely, for instance by the remote user via the display device 220, and more specifically by the input devices thereof, or locally, for example by the user of the headgear device 100.

The remote diagnostic platform 240 is composed of various diagnostic tools, which may include heart rate monitors, respiration monitors, blood sampling devices, other airway and/or fluid management devices, ultrasound equipment, ophthalmic equipment, and the like. The remote diagnostic platform 240 can be controlled remotely, for instance by the remote user via the display device 220, and more specifically by the input devices thereof, or locally, for example by the user of the headgear device 100.

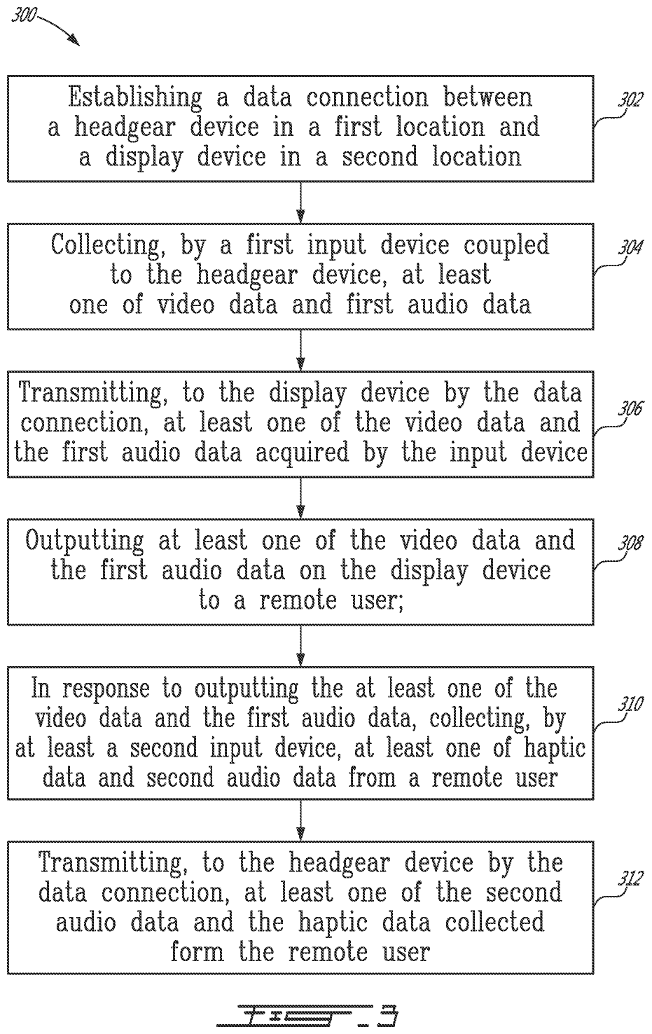

With reference to FIG. 3, the lifesaving trauma stabilization system 200 is configured for implementing a method 300 for providing lifesaving trauma stabilization medical telepresence to the remote user. At 302, a data connection is established between a headgear device in a first location, for example the headgear device 100, and a display device in a second location, for example the display device 220. In some embodiments, the first and second locations are different locations and are separated by a distance. The data connection may be established via the server box 210. The data connection may be established using any suitable communication protocols, for example packet-based protocols (e.g. TCP/IP) and the like. In some embodiments, the data connection is encrypted.

At 304, a first input device coupled to the headgear device 100, for example the AV capture device 110, collects at least one of video data and first audio data. The video data and the first audio data may be the aforementioned video data and local audio data collected by the AV capture device 110. The video data and the first audio data may be collected in any suitable format and at any suitable bitrate. As noted, the format and bitrate may be adjusted depending on various factors. For example, a low battery or weak signal condition may result in a lower bitrate being used.

At 306, at least one of the video data and the first audio data acquired by the AV capture device is transmitted to the display device 220 using the data connection, for example via server box 210. The server box 210 is configured for transmitting the video data and the first audio data to the display device using any suitable transmission protocols, as discussed hereinabove.

At 308, at least one of the video data and the first audio data is output on the display device 220 to the remote user. In some embodiments, the remote user is a doctor. The display device 220 may display the video data via one or more displays, and perform playback of the first audio data via one or more speakers. In some embodiments, the display device 220 includes a 3D-capable display for displaying 3D video collected by the AV capture unit 110, allowing the remote user to perceive depth in the 3D video via the display. In some embodiments, the display device 220 includes a surround-sound speaker system for performing playback of the first audio data.

At 310, in response to outputting the at least one of the video data and the first audio data, at least one of second audio data and haptic data, for example the aforementioned remote audio data and the haptic data, are collected from a remote user by a second input device, for example one or more of the input devices of the display device 220. The remote user may be a doctor, trauma surgeon, or any other suitable medical professional. For example, the display device 220 may include one or more microphones into which the remote user can speak to produce the remote audio data. In another example, the display device may include one or more buttons with which the remote user can interact to produce the haptic data. Still other examples are contemplated.

At 312, at least one of the second audio data and the haptic data collected from the remote user are transmitted to the headgear device 100 by the data connection, for example via the server box 210. The server box 210 is configured for transmitting the video data and the first audio data to the display device using any suitable transmission protocols, as discussed hereinabove. In some embodiments, due to the remote nature of the display device 220 from the server box 210 and the headgear device 100, the transmissions between the display device 220 and the server box 210 may occur via one or more data networks.

One or more additional operations may also be performed by or via the server box 210. In some embodiments, the server box 210 receives video data from the display device 220, or otherwise from the remote user, and causes the video data to be displayed for the user of the headgear device 100, for instance via the HUD 140. The video data can include one or more virtual-reality elements, one or more augmented-reality elements, and the like, which can, for example, be overlaid over the body of a patient being examined by the user of the headgear device 100.

In some other embodiments, the input devices of the display device 210 are also configured for collecting instructions for operating the remote robotic surgical platform 230 and/or for operating the remote diagnostic platform 240, for example from the remote user. The instructions can then be transmitted to the appropriate remote platform 230, 240, for instance via the server box 210, or via a separate connection. For example, the remote robotic surgical platform 230 and/or for operating the remote diagnostic platform 240 can be provided with cellular radios or other communication devices for receiving the instructions from the remote user, as appropriate.

Thus, by performing the method 300, audio and video data collected by the user of the headgear device 100 can be reproduced at a remote location for the remote user. In addition, the remote user can provide the user of the headgear device 100 with both audio- and haptic-based feedback. When used in a first responder context, a doctor or trauma surgeon in a remote location may provide detailed instructions to the first responder based on seeing exactly in high resolution and with critical depth perception what the first responder sees and hears on display device 220. In addition, instructions and/or other useful information can be presented to the first responder via the HUD 140, and the remote user can control the operation of the remote robotic surgical platform 230 and/or the remote diagnostic platform 240 while observing the state of the patient substantially in real-time.

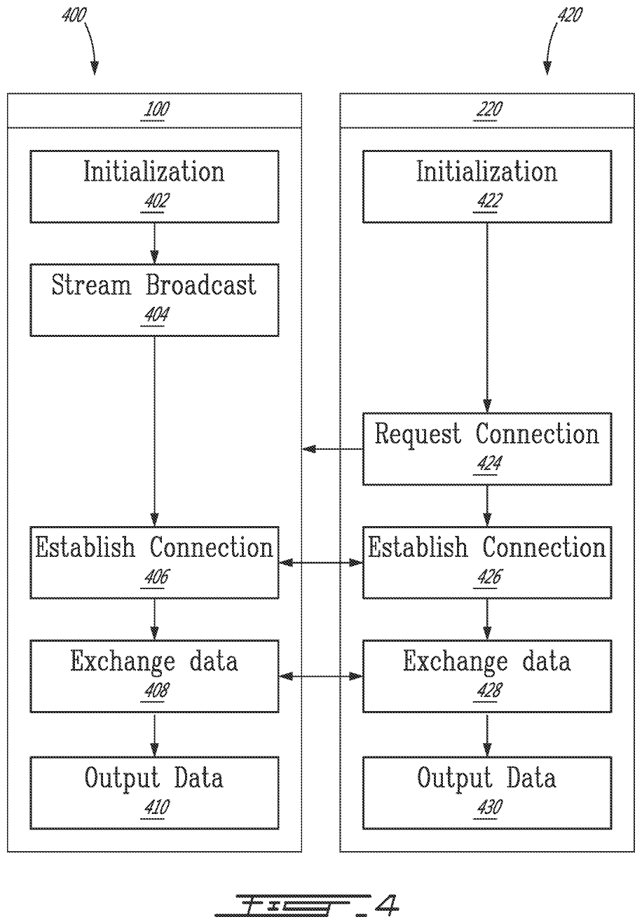

With reference to FIG. 4, a communication diagram for the lifesaving trauma stabilization medical telepresence system 200 is shown, with column 400 illustrating the steps performed at the headgear device 100 and column 420 illustrating the steps performed at the display device 220. Although certain steps are described herein as being performed at the headgear device 100 and/or at the display device 220, it should be noted that in some embodiments, some or all of certain steps may take place at the server box 210.

At 402, the headgear device 100 performs an initialization. This may include powering up various components, for example the AV capture device 110, and authenticating with one or more networks for transmission. At 422, the display device 220 performs an initialization, which may be similar to that performed by the headgear device 100.

At 404, the headgear device 100 begins to transmit an audio/video stream composed of the local audio data and the video data collected by the AV capture device 110. In some embodiments, this includes registration of the headgear device 100 and/or the stream produced thereby on a registry or directory. For example, the stream may be registered in association with an identifier of the user, an indication of the location at which the headgear device 100 is being used, or the like.

At 424, the display device 220 sends a request to establish a data connection with the headgear device 100. This can be performed using any suitable protocol, including any suitable handshaking protocol. Although 424 is shown as being performed by the display device 220, it should be noted that in certain embodiments the request to establish the data connection is sent by the headgear device 100 to the display device 220. For example, there may be a pool of doctors which are available to be contacted by the first responder, and the headgear device 100 may submit a request to be assigned to one of the first available doctors of the pool of doctors and trauma surgeons as may be found in the Emergency Room of a Regional Trauma Centre.

At 406 and 426, the data connection is established between the headgear device 100 and the display device 220. At 408 and 428, data is exchanged between the headgear device 100 and the display device 220. This includes the headgear device 100 sending the video data and the local audio data to the display device 220, and the display device 220 sending the remote audio data and the haptic data to the headgear device 100. In some embodiments, additional data, for example for controlling the cameras 112, 114 of the headgear device 100 or for displaying on a HUD of the headgear device 100 is also exchanged.

At 410 and 430, the data exchanged at 408 and 428 is output. At the headgear device 100, this may include performing playback of the remote audio data via the speakers 120, and outputting the haptic data via the haptic system 130. At the display device 220, this may include displaying the video data and performing playback of the local audio data via one or more screens and one or more speakers, respectively. In embodiments where additional data is exchanged, 410 further includes displaying information on the HUD 140 and/or moving the cameras 112, 114.

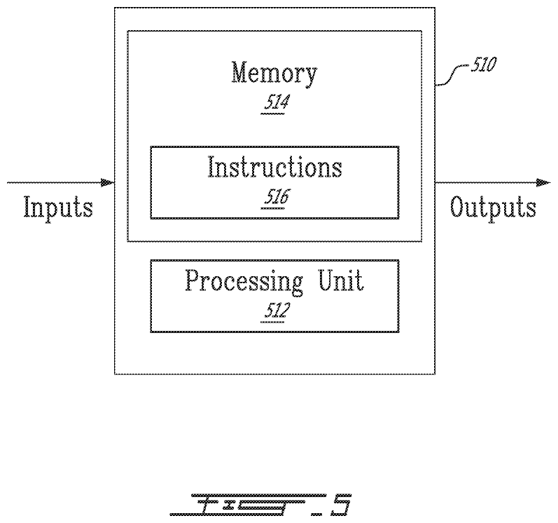

With reference to FIG. 5, the method 300 and/or the actions shown in the communication diagram 400 may be implemented by a computing device 510, comprising a processing unit 512 and a memory 514 which has stored thereon computer-executable instructions 516. The server box 210 and/or the display device 220 may be embodied as or may comprise an embodiment of the computing device 510.

The processing unit 512 may comprise any suitable devices configured to implement the method 300 and/or the actions shown in the communication diagram 400 such that instructions 516, when executed by the computing device 510 or other programmable apparatus, may cause performance of some or all of the method 300 and/or the communication diagram 400 described herein. The processing unit 512 may comprise, for example, any type of microprocessor or microcontroller, a digital signal processing (DSP) processor, a central processing unit (CPU), an integrated circuit, a field programmable gate array (FPGA), a reconfigurable processor, other suitably programmed or programmable logic circuits, or any combination thereof.

The memory 514 may comprise any suitable known or other machine-readable storage medium. The memory 514 may comprise non-transitory computer readable storage medium, for example, but not limited to, an electronic, magnetic, optical, electromagnetic, infrared, or semiconductor system, apparatus, or device, or any suitable combination of the foregoing. The memory 514 may include a suitable combination of any type of computer memory that is located either internally or externally to device, for example random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. Memory 514 may comprise any storage means (e.g., devices) suitable for retrievably storing machine-readable instructions 516 executable by processing unit 512.

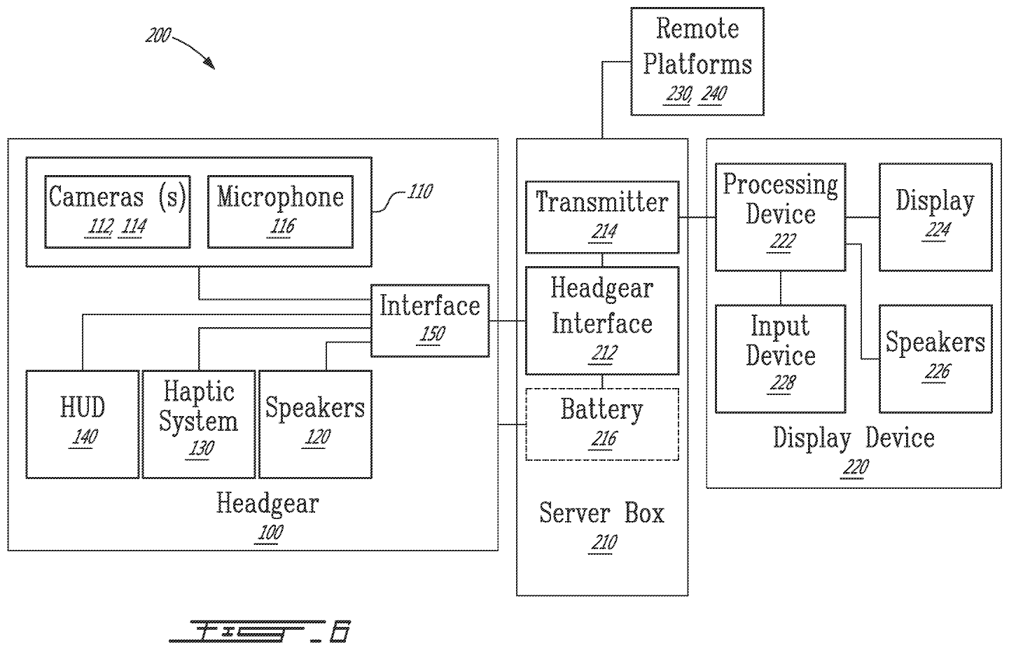

With reference to FIG. 6, there is shown an embodiment of the lifesaving trauma stabilization medical telepresence system 200, which includes the headgear device 100, the server box 210, and the display device 220. The headgear device 100, as depicted, includes the AV capture device 100, the speakers 120, the haptic system 130, and the interface 150. The AV capture device includes one or more cameras, including at least one of the cameras 112, 114, and the microphone 116. In some embodiments, the interface 150 is configured for establishing the data connection with the server box 210 and for processing the remote audio data and the haptic data sent from the display device to the headgear device 100. The interface 150 sends the processed remote audio data and haptic data to the speakers 120 and the haptic system 130, respectively, for playback to the user of the headgear device 100.

In some embodiments, the server box 210 comprises a headgear interface 212, a transmitter 214, and optionally a battery 216 or other power source. The headgear interface 212 is configured for establishing the data connection with the headgear device 100, for example via the interface 150. The headgear interface 212 may communicate with the headgear device 100 over a wired or wireless connection, using any suitable protocol, as described hereinabove. In some embodiments, the interface 150 and the headgear interface 212 establish the data connection over a USB™-based connection. In other embodiments, the interface 150 and the headgear interface 212 establish the data connection over a Zigbee™-based connection.

The transmitter 214 is configured for establishing the data connection between the server box 210 and the display device 220. Once the interface 150-headgear interface 212 connection and the transmitter 214-display device 220 connections are established, the data connection between the headgear device 100 and the display device 220 is established. The transmitter may be a wireless transmitter, for example using one or more cellular data technologies.

The battery 216 is configured for providing electrical power to the headgear device 100. The battery 216 may provide any suitable level of power and any suitable level of autonomy for the headgear device 100. In some embodiments, the battery 216 is a lithium-ion battery. In embodiments where the server box 210 includes battery 216, the server box 216 includes a charging port for recharging the battery 216 and/or a battery release mechanism for replacing the battery 216 when depleted.

In this embodiment, the display device 220 includes a processing device 222, a display 224, speakers 226, and input devices 228. The processing device 222 is configured for establishing the data connection with the server box 210 and for processing the video data and the local audio data sent by the headgear device 100. The processed video and local audio data is sent to the display 224 and the speakers 226, respectively, for playback to the remote user. In some embodiments, the processing device 222 includes one or more graphics processing units (GPUs).

The display 224 may include one or more screens. The screens may be televisions, computer monitors, projectors, and the like. In some embodiments, the display 224 is a virtual reality or augmented reality headset. In some embodiments, the display 224 is configured for displaying 3D video to the remote user. The speakers 226 may be any suitable speakers for providing playback of the local audio data. In some embodiments, the speakers 226 form a surround-sound speaker system.

The input devices 228 are configured for receiving from the remote user at least one of remote audio data and haptic data. The input devices may include one or more microphones, a keyboard, a mouse, a joystick, a touchscreen, and the like, or any suitable combination thereof. In some embodiments, a dedicated input device is provided for inputting haptic data, for example a replica of the headgear device 100 with input buttons or controls which mirror the locations of the elements of the haptic system 130 on the headgear device 100.

In some embodiments, the headgear device 100, server box 210, and/or the display device 220 is configured for recording and/or storing at least some of the video data, the local audio data, the remote audio data, and the haptic data. For example, the server box 210 further includes a hard drive or other storage medium on which the video data and the local audio data is stored. In another example, the display device 220 has a storage medium which stores the video data, the local audio data, the remote audio data, and the haptic data. In some embodiments, the headgear device 100 and/or the display device 220 is configured for replaying previously recorded data, for example for use in training simulations, or when signal strength is weak and transmission is slow or impractical.

The methods and systems for providing lifesaving trauma stabilization medical telepresence to a remote user described herein may be implemented in a high level procedural or object oriented programming or scripting language, or a combination thereof, to communicate with or assist in the operation of a computer system, for example the computing device 510. Alternatively, the methods and systems described herein may be implemented in assembly or machine language. The language may be a compiled or interpreted language. Program code for implementing the methods and systems described herein may be stored on a storage media or a device, for example a ROM, a magnetic disk, an optical disc, a flash drive, or any other suitable storage media or device. The program code may be readable by a general or special-purpose programmable computer for configuring and operating the computer when the storage media or device is read by the computer to perform the procedures described herein. Embodiments of the methods and systems described herein may also be considered to be implemented by way of a non-transitory computer-readable storage medium having a computer program stored thereon. The computer program may comprise computer-readable instructions which cause a computer, or more specifically the processing unit 512 of the computing device 510, to operate in a specific and predefined manner to perform the functions described herein, for example those described in the method 300 and the communication diagram 400.

Computer-executable instructions may be in many forms, including program modules, executed by one or more computers or other devices. Generally, program modules include routines, programs, objects, components, data structures, etc., that perform particular tasks or implement particular abstract data types. Typically the functionality of the program modules may be combined or distributed as desired in various embodiments.

The above description is meant to be exemplary only, and one skilled in the art will recognize that changes may be made to the embodiments described without departing from the scope of the invention disclosed. Still other modifications which fall within the scope of the present invention will be apparent to those skilled in the art, in light of a review of this disclosure.

Various aspects of the methods and systems for described herein may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments. Although particular embodiments have been shown and described, it will be obvious to those skilled in the art that changes and modifications may be made without departing from this invention in its broader aspects. The scope of the following claims should not be limited by the embodiments set forth in the examples, but should be given the broadest reasonable interpretation consistent with the description as a whole.

1. A method for providing a lifesaving trauma stabilization medical telepresence to a remote user, comprising:
establishing a data connection between a lifesaving trauma stabilization helmet associated with a first user in a first location and a display device in a second location, the first location being remote from the second location;
collecting, by a first input device communicatively integrated to the lifesaving trauma stabilization helmet, first video data and first audio data;
transmitting, to the display device by the data connection, the first video data and the first audio data acquired by the input device;
outputting the first video data and the first audio data on the display device to the remote user;
in response to outputting the first video data and the first audio data, collecting contextual information for the first user from the remote user using a second input device located in the second location;
transmitting, to the lifesaving trauma stabilization helmet by the data connection, the contextual information collected from the remote user; and
presenting the contextual information to the first user as haptic feedback using a haptic output device comprising two groups of three vibrating elements having unique tones integrated to opposing lateral sides of the lifesaving trauma stabilization helmet, wherein presenting the contextual information to the first user as haptic feedback comprises producing a vibration pattern by causing at least some vibrating elements of the two groups of three vibrating elements to produce vibrations.

2. The method of claim 1, wherein the first video data comprises three-dimensional video data, wherein outputting the first video data comprises outputting the three-dimensional video data via at least one three-dimension-capable display.

3. The method of claim 1, wherein the first audio data comprises surround-sound audio data, wherein outputting the first audio data comprises outputting the surround-sound audio data via at least one surround-sound playback system.

4. The method of claim 1, further comprising:
transmitting, to the lifesaving trauma stabilization helmet by the data connection, second video data associated with a particular medical situation; and
displaying the second video data on a head-up display of the lifesaving trauma stabilization helmet.

5. The method of claim 4, wherein displaying the second video data on the head-up display of the lifesaving trauma stabilization helmet comprises displaying at least one augmented reality element on the head-up display.

6. The method of claim 5, wherein the at least one augmented reality element is overlain over a body of a patient within a field-of-view of the head-up display.

7. The method of claim 1, wherein collecting the first video data comprises collecting video of a remote robotic surgical platform, the method further comprising:
collecting, by at least the second input device, instructions for operating the remote robotic surgical platform; and
transmitting the instructions to the remote robotic surgical platform.

8. The method of claim 1, wherein collecting the first video data comprises collecting video of a remote diagnostic platform, the method further comprising:
collecting, by at least the second input device, instructions for operating the remote diagnostic platform;
transmitting, by the data connection, the instructions to the remote diagnostic platform;
obtaining diagnostic information from the remote diagnostic platform; and
transmitting, by the data connection, the diagnostic information to the display device.

9. The method of claim 8, wherein the remote diagnostic platform comprises an ultrasound equipment.

10. The method of claim 8, wherein the remote diagnostic platform comprises an ophthalmic equipment.

11. A system for providing lifesaving trauma stabilization, the system comprising:
a processor;
a memory storing computer-readable instructions;
a network interface;
a lifesaving trauma stabilization helmet configured for mounting to a head of a first user, the lifesaving trauma stabilization helmet comprising:
at least one camera configured to capture first video data;
at least one microphone configured to capture first audio data;
at least one speaker; and
a haptic output device comprising two groups of three vibrating elements having unique tones integrated to opposing lateral sides of the helmet;
wherein the computer-readable instructions are executable by the processor for:
transmitting, by the network interface, the first video data and the first audio data to a remote display device configured to output the first video data and the first audio data to the remote user in a second location, the first location being remote from the second location;
obtaining, by the network interface, contextual information for the first user, the contextual information collected from the remote user using a remote input device located in the second location; and
in response to obtaining the contextual information for the first user, presenting the contextual information to the first user as haptic feedback by causing at least some vibrating elements of the two groups of three vibrating elements to produce vibrations.

12. The system of claim 11, wherein the at least one camera comprises two cameras configured to collect three-dimensional video data, wherein the computer-readable instructions cause the processor to transmit the three-dimensional video data to a three-dimension-capable remote device.

13. The system of claim 11, wherein the at least one microphone is an array of microphones configured to collect surround-sound audio data, wherein the computer-readable instructions cause the processor to transmit the surround-sound audio data to a surround-sound-capable remote device.

14. The system of claim 11, wherein the lifesaving trauma stabilization helmet further comprises a head-up display, wherein the computer-readable instructions further cause the processor to:
obtain second video data associated with a particular medical situation; and
display the second video data on the head-up display of the lifesaving trauma stabilization helmet.

15. The system of claim 14, wherein displaying the second video data on the head-up display of the lifesaving trauma stabilization helmet comprises displaying at least one augmented reality element on the head-up display.

16. The system of claim 15, wherein the at least one augmented reality element is overlain over a body of a patient within a field-of-view of the head-up display.

17. The system of claim 11, further comprising a remote robotic surgical platform coupled to the lifesaving trauma stabilization helmet, wherein the at least one camera is configured to capture the first video data which comprises video of the remote robotic surgical platform, wherein the computer-readable instructions further cause the processor to:
obtain instructions for operating the remote robotic surgical platform; and
transmit the instructions to the remote robotic surgical platform.

18. The system of claim 11, further comprising a remote diagnostic platform coupled to the lifesaving trauma stabilization helmet, wherein the at least one camera is configured to capture the first video data which comprises video of the remote diagnostic platform, wherein the computer-readable instructions further cause the processor to:
obtain instructions for operating the remote diagnostic platform;
transmit the instructions to the remote diagnostic platform;
obtain diagnostic information from the remote diagnostic platform; and
transmit the diagnostic information to the remote device.

19. The system of claim 18, wherein the remote diagnostic platform comprises an ultrasound equipment.

20. The system of claim 18, wherein the remote diagnostic platform comprises an ophthalmic equipment.
